(19) United States
(12) Patent Application Publication
(10) Pub. No.: US 2002/0182157 A1
(43) Pub. Date: Dec. 5, 2002

(54) Lipstick and lip liner in one

(76) Inventor: Suzanne Grayson, San Juan Capistrano, CA (US)

Correspondence Address: FISH & NEAVE, 1251 AVENUE OF THE AMERICAS, 50TH FLOOR, NEW YORK, NY, 10020-1105, US

(21) Appl. No.: 10/078,054

(22) Filed: Feb. 15, 2002

Related U.S. Application Data
(60) Provisional application No. 60/269,306, filed on Feb. 15, 2001.

(51) Int. Cl. A61K 7/025; A61K 6/00

(52) U.S. Cl. 424/64; 424/401

(57) ABSTRACT

The present invention relates lip cosmetics having an elongated lipstick core and an outer layer of lip liner around the periphery of the elongated lipstick core and to methods for preparing and using such cosmetics.

### TECHNICAL FIELD OF THE INVENTION

This invention relates to lip cosmetics. More particularly, this invention relates to lip cosmetics having an elongated lipstick core and an outer layer of a lip liner composition and to methods for making and using such cosmetics.

### BACKGROUND OF THE INVENTION

Lipstick is used to enhance the shape and/or the color of the lips. Lipstick is preferably long wearing. At the same time, lipstick is preferably moisturizing. Additionally, lipstick preferably should cleanly outline the lips and not migrate beyond the natural outline of the lips to which it is applied. The migration of lipstick beyond the area of the lips to which it is applied is sometimes referred to as “feathering.”

There is no lipstick product that provides an optimum combination of the above benefits. A consumer must, therefore, choose some benefits at the expense of other benefits. For example, the consumer can use a creamy, moisturizing lipstick for good feel and good conditioning. However, such lipsticks have relatively low wear and tend to feather. Another option is to use a drier lipstick having increased wear and reduced feathering. Such drier lipsticks, however, tend to make the lips feel and look dry.

A third option is to use a separate lip liner product in addition to a lipstick product. Such lip liner products are designed to be applied to the outline of the lips. One benefit of using a lip liner product is to prevent or minimize substantially the feathering of a lipstick product that is applied to the lips. Other benefits of using a lip liner include providing an attractive outline to the lips or reshaping the lips, and/or providing a portion of long-lasting color to the outline of the lips.

A lipstick and a lip liner in one product would be desirable because of the convenience of having the benefits of a lipstick and a lip liner in one product. One known combination lipstick and lip liner product is described in Kapustin, U.S. Pat. No. 4,740,097, which has a lip liner formulation positioned along a small portion of one side of a lipstick bullet. The Kapustin product is said to have a “line of demarcation” between the lip liner and lipstick so that “little mixing” of the lip liner and lipstick occurs upon application to the lips.

The Kapustin product, however, is difficult to use. This is so because of the amount of manipulation that is required to outline the lips with lip liner adequately. For example, after applying the Kapustin product to the upper lip portion, the product would have to be turned about 180 degrees to apply the product to the lower lip.

It is therefore an object of the present invention to provide a lip cosmetic that is a lipstick and lip liner in one and that is more convenient to use than the Kapustin product.

It is also an object of the present invention to provide a lip cosmetic that is more convenient to use than the Kapustin product and that has a lipstick core surrounded by a lip liner composition.

It is also an object of the present invention to provide methods for preparing and using such lip cosmetics.

### SUMMARY OF THE INVENTION

The lip cosmetics of the present invention comprise an elongated lipstick core and an outer layer of lip liner around the periphery of the elongated lipstick core. Preferably, the lip cosmetic and the lipstick core are cylindrical throughout at least the major portion of their length. In such embodiments, the radius of the lip cosmetic is preferably no more than 1.4 times the radius of the lipstick core. More preferably, the radius of the lip cosmetic is no more than about 1.2 times the radius of the lipstick core.

The lipstick core composition is present in an amount such that the lip cosmetic contains at least about 50% by weight of lipstick.

Preferred embodiments include: (a) lip cosmetics that contain from about 85% to about 95% by weight of the lipstick core and about 5% to about 15% by weight of the lip liner, and (b) lip cosmetics that contain about 50% to about 65% of the lipstick core and about 35% to about 50% of the lip liner.

The lip cosmetics of this invention may be made by a method comprising the steps of:

providing a mold having an internal volume generally corresponding to the exterior shape of the lip cosmetic;

inserting into the mold a lipstick core mandrel having a shape generally corresponding to the shape of the lipstick core, leaving a lip liner volume;

introducing liquified lip liner composition into the lip liner volume and solidifying the lip liner composition to form what becomes the coating of lip liner;

removing the lipstick core mandrel from the mold leaving a lipstick core volume; and

introducing liquified lipstick composition into the lipstick core volume and solidifying the lipstick composition.

The lip cosmetics of this invention may also be made by a method comprising the steps of:

providing a first mold having an internal volume generally corresponding to the lipstick core;

introducing liquified lipstick composition into the volume generally corresponding to the lipstick core and solidifying the lipstick composition to form the lipstick core;

providing a second mold having an internal volume generally corresponding to the lip cosmetic;

positioning the lipstick core into the internal volume generally corresponding to the lip cosmetic, leaving a lip liner volume; and

introducing liquified lip liner composition into the lip liner volume and solidifying the lip liner composition to form the coating of lip liner around the lipstick core.

In one embodiment of the method described in the preceding paragraph, the first mold comprises a casing having an internal volume generally corresponding to the exterior shape of the lip cosmetic and a lip liner mandrel having a shape generally corresponding to the shape of the lip liner that is inserted into the casing, thereby leaving a lipstick core volume. In this method, liquified lipstick composition is then introduced into the lipstick core volume and solidified. The lip liner mandrel is then removed from the casing leaving a lip liner volume. Then liquified lip liner composition is introduced into the lip liner volume and solidified, thereby leaving the lip cosmetic in the casing.

The lip cosmetic of this invention may be used by applying the lip cosmetic to the lips. In its intended use, the lip cosmetic is applied to the lip area of the user in such a way that the lip liner composition outlines the lips, thereby preventing or minimizing the tendency of the lipstick that was applied to the lips to feather outside the boundary of the lips and/or highlighting the outline of the lips. Because the lip liner is around the entire periphery of the lipstick core, this can be accomplished relatively simply and with minimal manipulation of the lip cosmetic.

### BRIEF DESCRIPTION OF THE DRAWINGS

The above and other objects and advantages of the invention will be apparent upon consideration of the following detailed description, taken in conjunction with the accompanying drawings, in which like reference characters refer to like parts throughout, and in which:

FIG. 1 is a perspective view of a lip cosmetic of the invention.

FIG. 2 is a plan view of the lip cosmetic of FIG. 1.

FIG. 3 is a plan view of an oval lip cosmetic of the invention.

FIG. 4 is an elevational view of a lip cosmetic of the invention with a bullet shaped applicator end that has not been beveled.

FIG. 5 is a side elevational view of a lip cosmetic of the invention with a fishtail shaped applicator end that has not been beveled.

FIG. 6 is a front elevational view of the lip cosmetic of FIG. 5.

FIG. 7 is a side elevational view of a lip cosmetic of the invention with a fishtail shaped applicator end that has been beveled.

FIG. 8 is a front elevational view of the lip cosmetic of FIG. 7.

FIG. 9 is a cross-sectional view of a mold assembly, including a lipstick core mandrel, that illustrates one embodiment of a mold assembly according to the invention.

FIGS. 10 and 11 are front and side elevational views, respectively, of a lip cosmetic of the invention in a relatively long, slim casing.

### DETAILED DESCRIPTION OF THE INVENTION

The present invention provides lip cosmetics that are a lipstick and a lip liner in one and methods for making and using such cosmetics. The lip cosmetics comprise an elongated lipstick core and an outer layer of lip liner around the lipstick core.

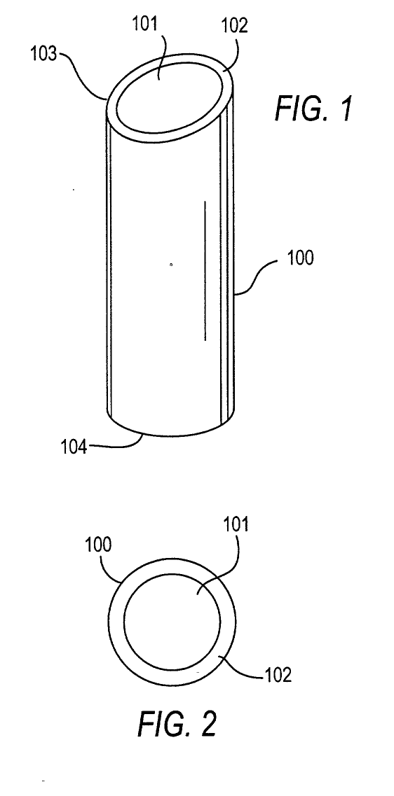

Both the lip cosmetic and the lipstick core may have any shape that is convenient to apply to the lip area. Preferably, the lip cosmetic is cylindrical along at least the major portion of its length, as depicted in FIG. 1. Such a cylindrical lip cosmetic would appear from a top view, as depicted in FIG. 2. As appreciated by skilled practitioners, a typical cylindrical lip cosmetic has a diameter of about 7/16 inches. This diameter may, however, vary. Preferably, the lip cosmetic of this invention has a diameter of about 7/16 inches.

The lip cosmetic of the invention has a base end that desirably is mounted in a casing of the type that is conventionally used in commercial lip cosmetic products. Such conventional casings contain a mechanism to move the applicator end of the lip cosmetic to a position extending outside the casing, thereby permitting the application of the lip cosmetic to the lip area. In a cylindrical lip cosmetic, such a mechanism would comprise a cup having a diameter of about 0.5 inches for mounting the cosmetic. Suitable casings for the storage and application of lip cosmetics are well known in the art and are not part of the present invention.

As shown in FIG. 1, the applicator end of the lip cosmetic is preferably beveled for easy application to the skin. The applicator end is beveled at up to about 45°, preferably about 30° to about 45°, relative to the lip cosmetic. Alternatively, it can be beveled at other angles as desired. The bevel shape of the applicator end may be obtained by various methods well known in the art. Preferably, the bevel shaped end is formed as part of the molding process by which the lip cosmetic is made, as is described more fully below. Alternatively, the bevel shaped end may be formed after the molding process, e.g., by clipping the applicator end of the lip cosmetic into the desired shape after the molding process.

A preferred lip cosmetic of the invention is depicted in FIGS. 1 and 2 and is designated 100. The outer layer of lip liner is designated 102 and the elongated lipstick core is designated 101. As shown in FIG. 1, the end 103 of the lip cosmetic that is to be applied to the lips is beveled. The opposite end 104 is preferably mounted in a casing (not shown) of the type previously discussed.

Preferably, the lipstick composition is present in an amount such that the lip cosmetic contains at least about 50% by weight of lipstick. In specific embodiments, the lipstick core and lip liner may be present in amounts such that (a) the lip cosmetic contains from about 85% to about 95% by weight of lipstick and about 5% to about 15% by weight of lip liner, or (b) the lip cosmetic contains from about 50% to about 65% by weight of lipstick and about 35% to about 50% by weight of lip liner.

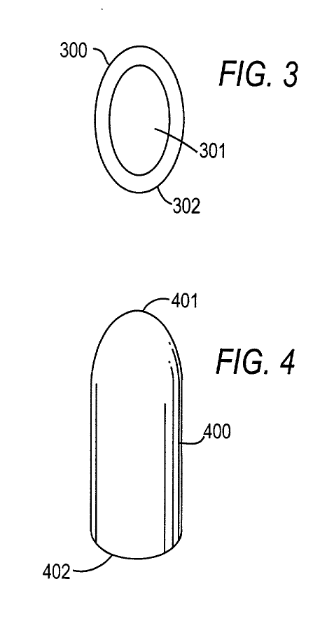

Preferably, the lip cosmetic and the lipstick core are cylindrical throughout at least the major portion of their length, as shown in FIG. 1. If the lip cosmetic and the lipstick core are cylindrical throughout at least a major portion of their length, the radius of the lip cosmetic is desirably no more than about 1.4 times (preferably no more than about 1.2 times) the radius of the lipstick core. Other shapes may be used however. For example, the lip cosmetic and lipstick core may have a teardrop, oval, square, or hexagonal shape throughout at least a major portion of their length. An oval shaped lip cosmetic of the invention is depicted in FIG. 3. Lip cosmetic 300 includes outer layer 302 and lipstick core 301. Lip cosmetic 300 is preferably mounted in an oval casing (not shown).

Prior to beveling, the applicator end of the lip cosmetic may have various shapes that are known in the art, such as a bullet shape or a fishtail shape.

A lip cosmetic having a bullet shaped applicator end is depicted in FIG. 4. The shape of cosmetic 400, including the applicator end 401, appears the same from all views around the perimeter of cosmetic 400. End 402 is the base end.

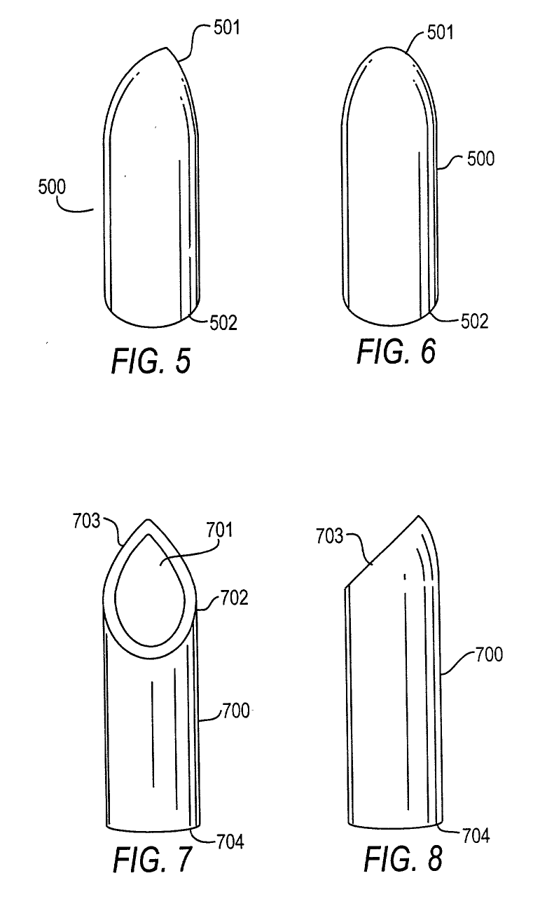

A lip cosmetic having a fishtail shaped applicator end is depicted in FIGS. 5 and 6. FIG. 5 is a side view of cosmetic 500, applicator end 501, and base end 502, while FIG. 6 is a front view. A rear view would appear generally the same as the front view. End 502 is the base end.

These various shapes may be obtained by methods well known in the art. For example, a fishtail shaped end is usually formed as part of the molding process by which the lip cosmetic is made, as is described more fully below.

A lip cosmetic of the invention having a beveled fishtail applicator end is depicted in FIG. 7 (side view) and FIG. 8 (front view). Lip cosmetic 700 includes outer layer 702 and elongated lipstick core 701. As shown in FIG. 7, beveled end 703, which is to be applied to the lips, is teardrop shaped. Opposite end 704 is preferably mounted in a casing (not shown) of the type previously discussed. The end may also be beveled on the front face as desired.

The lipstick core may be made from any lipstick composition, including commercially available lipstick compositions. Thus, the lipstick compositions that may be used as the lipstick core in this invention include any complete lipstick compositions. Such lipstick compositions typically comprise a base material, and, optionally, a colorant and/or other cosmetic materials. The ingredients that may be used in such compositions are well known to skilled practitioners and are described, for example, in CFTA International Cosmetic Ingredient Dictionary, 4th ed., J. M. Nikitakis et al., eds., The Cosmetic, Toiletry and Fragrance Association, Washington, D.C. (1991), which is incorporated herein by reference. The lipstick compositions used in this invention may, for example, be mixtures that contain waxes, emollients, emulsifiers, pigments, and/or dyes. As appreciated by skilled practitioners, certain ingredients may be used for more than one purpose. Titanium dioxide, for example, may be used as both a colorant and a sunscreen.

Similarly, the coating of lip liner may be made from any lip liner composition, including commercial lip liners. Lip liner compositions typically comprise a base material, and, optionally, a colorant and/or other cosmetic materials. The ingredients that are used to make lip liner compositions are well known to skilled practitioners and are described, for example, in CFTA International Cosmetic Ingredient Dictionary, supra. Thus, the lip liner compositions used in this invention may, for example, be mixtures that contain waxes, emollients, emulsifiers, pigments, and/or dyes.

The lip liner and the lipstick for use in the lip cosmetic of the present invention are defined so that in the lip cosmetic's intended use, the lip liner serves one or both of the following two purposes: (1) the lip liner has the same or a different color than the lipstick and prevents or minimizes the tendency of the lipstick in the lip cosmetic to feather outside the borders of the lips outlined by the lip liner and (2) the lip liner has a different color than the lipstick, thereby highlighting the outline of the lips in the intended use of the lip cosmetic. Preferably, the lip liner and lipstick are selected so that both purposes are served. Typically the lip liner is “drier” than the lipstick composition in the lip cosmetic, i.e., the lip liner composition has a higher melting point than the lipstick composition. In addition, the lip liner is typically firmer, less slippery, and has less shine than the lipstick composition used in the lip cosmetic.

Suitable cosmetic base materials that may be used in the lipstick core or lip liner include waxes. An acceptable wax can be a solid or semi-solid wax having a melting point of 30 to 120° C. and generally includes animal waxes, plant waxes, mineral waxes, silicone waxes, synthetic waxes, and petroleum waxes. Waxes include, for example, bayberry, beeswax, candelilla, carnauba, ceresin, spermaceti, cetyl esters, stearyl esters, hydrogenated cottonseed oil, hydrogenated castor oil, hydrogenated palm oil, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, PEG beeswax, PEG-6 beeswax, PEG-8 beeswax, PEG-20 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, polyethylene, stearoxy dimethicone, dimethicone behenate, glyceryl tribehenate, and stearyl dimethicone, as well synthetic homo- and copolymer waxes such as PVP/eicosene copolymer and PVP/hexadecene copolymer.

Other suitable cosmetic base materials that may be used in the lipstick or lip liner include cosmetics oils. Suitable oils include, for example, esters of the formula RC(O)OR′ wherein R and R′ are each independently a C₁₋₂₅, preferably a C₄₋₂₀ straight or branched chain alkyl or alkenyl, optionally substituted by alkoxycarbonyl, alkylcarbonyloxy, or hydroxy. Such esters include, for example, isotridecyl isononanoate, PEGA4 diheptanoate, isostearyl neopentanoate, tridecyl neopentanoate, cetyl octanoate, isopropyl palmitate, cetyl palmitate, cetyl ricinoleate, cetyl stearate, isopropyl myristate, cetyl myristate, cetyl lactate, cetyl isooctanoate, stearyl isooctanoate, coco-dicaprylate/caprate, decyl isostearate, isodecyl oleate, octyldodecyl oleate, isodecyl neopentanoate, isohexyl neopentanoate, octyl palmitate, dioctyl malate, tridecyl octanoate, myristyl myristate, octyldodecyl myristate, myristyl lactate, butyl stearate, octyldodecyl ricinoleate, propylene glycol monolaurate, 2-ethylhexyl succinate, glyceryl tri-2-ethylhexanate, and fatty alcohols such as oleyl alcohol, isocetyl alcohol, octododecanol, hexadecyl alcohol, and fatty acids, such as oleic acid, as well as the esters disclosed in CFTA International Cosmetic Ingredient Dictionary, supra.

Other suitable oils include (a) glyceryl esters of fatty acids, or triglycerides, such as, for example, castor oil, lanolin oil, triisocetyl citrate, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, and walnut oil; (b) nonvolatile hydrocarbons such as, for example, isoparaffins, hydrogenated polyisobutene, polybutene, mineral oil, squalene, and petrolatum; (c) nonvolatile nonfluorinated silicones; (d) fluorinated oils such as, for example, fluorinated silicones or perfluoropolyether; and (e) sorbitan derivatives such as, for example, PEG sorbitan beeswax, PEG sorbitan isostearate, PEG sorbitan lanolate, PEG sorbitan laurate, PEG sorbitan oleate, PEG sorbitan palmitate, PEG sorbitan stearate, polysorbates, sorbitan trioleates, sorbitan sesquioleates, sorbitan stearates, and sorbitan tristearates. Other materials may be used in the present invention including cocoa butter, lanolin, and lanolin alcohol, and various derivatives thereof, such as, for example, acetylated lanolin and acetylated lanolin alcohol.

Suitable cosmetic base materials often contain at least one hardening agent. Hardening agents include, for example, semirefined, full refined and/or synthetically derived paraffins with melting points from about 45 to about 120° C. (for example, the Eskar® series manufactured by the Amoco Oil Company, Chicago, Ill. and the Parvan® series available from Exxon Company, Houston, Tex.), microcrystalline waxes with melting points from about 60 to about 95° C. (for example, the Be Square® series from the Bareco Division of Petrolite Corporation and the Multiwax® series from Witco Chemical Corporation, Sonneborn Division, New York, N.Y.) and certain synthetically derived, modified hydrocarbon waxes, such as the Vestowaxes® and Duroxon® waxes available from Astor Wax Corporation, Harrison, N.Y. Hardening agents, when used, are used in amounts sufficient to (a) provide the proper hardness so that the composition can be formed into a stick, and (b) provide a texture that is smooth and uniform and enables the composition to be deposited smoothly on the lips when the lip cosmetic is applied to the lips.

The lipstick core and lip liner compositions of the invention may also contain dry particulate matter having a particle size of about 0.02 to about 200, preferably about 0.5 to about 100, microns. The particulate matter may be colored or non-colored. Suitable powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, and walnut shell powder. The above mentioned powders may be surface treated with lecithin, amino acids, mineral oil, silicone oil or various other agents either alone or in combination, which coat the powder surface and render the particles more lipophilic in nature.

The powder component also may comprise various organic and inorganic pigments. Powdered organic pigments are generally various aromatic types including azo, indigoid, triphenylmethane, anthraquinone, and xanthine dyes which are designated as D&C and FD&C blues, browns, greens, oranges, reds, yellows, etc. Organic pigments generally consist of insoluble metallic salts of certified color additives, referred to as the Lakes, in particular the Lakes of D&C and FD&C colors. Powdered inorganic pigments include iron oxides, titanium oxides, calcium carbonates, ultramarines, chromium, and chromium hydroxide colors.

Each of the lipstick core and the coating of lip liner desirably contain at least one colorant in the cosmetic base material. Preferably, both the lipstick core and the coating of lip liner contain at least one colorant. Such colorants are well known to those of ordinary skill in the art. They include, for example, inorganic and organic dyes or pigments, lipophilic dyes, lakes, micas, and pearls. Preferably, the colorant comprises an organic dye, an inorganic dye, or a mixture thereof. More preferably, the colorant comprises a lipophilic dye, a lake, a pigment, or a mixture thereof. Two or more suitable colorants may be mixed to provide a desired shade.

Colorants may be divided into two classes. The first class includes stains and organic dyes. Stains and organic dyes are generally thinner materials than the inorganic dyes and pigments and have a tendency to stain the lips when used in a lip cosmetic. The characteristics of the stains and organic dyes make them more suited for use in the lipstick core composition.

The second class includes inorganic dyes and pigments. The inorganic dyes and pigments are generally drier and more rigid materials. The characteristics of the inorganic dyes and pigments make them more suited for use in the lip liner composition.

The colorant materials and pearling agents presently used in commercial lipstick compositions are suitable for use in the compositions of the present invention. Among these are the lakes of FD&C Red #3, D&C Red #3, D&C Red #6, D&C Red #7, D&C Red #9, D&C Red #19, D&C Red #21, D&C Red #27, D&C Red #30, D&C Red #33, D&C Red #36, D&C Red #40, D&C Orange #5, D&C Orange #17, FD&C Yellow #5, FD&C Yellow #6, D&C Yellow #6, D&C Yellow #10, D&C Yellow #11, FD&C Blue #1, D&C Blue #1, D&C Blue #6, D&C Violet #2, iron oxides, carmine, mica, titanium dioxide, titanium dioxide coated mica, bismuth oxychloride, guanine, zinc oxide, talc, and kaolin.

Suitable colorant materials may be derived from natural sources such as plants, animals, or minerals. Such colorant materials include, for example, cochineal extract, alkanet, annatto, carotene, chlorophyll, saffron, turmeric, beet juice powder, carmine, caramel, grape skin extract, and henna.

If a vehicle for the colorant is used, the vehicle must be compatible with the other components of the composition, i.e., it must be miscible with or soluble in the components and it should not synerize (sweat) on the surface of the finished product. Among such suitable vehicles are squalane, isopropyl myristate, butyl stearate, decyl oleate, isopropyl palmitate, 2-ethyl hexylstearate, oleyl alcohol, octyl dodecanol, isocetyl alcohol and erucyl erucate. A mixture of suitable vehicles may also be used.

In a preferred embodiment of this invention, the lipstick core and the lip liner contain the same colorant. In another preferred embodiment, the lipstick core and the lip liner contain different colorants.

The “color value” of a composition, as used herein, refers to the percent by weight of colorant in the composition. The coating of lip liner of this invention may have the same, a lesser, or a greater color value than the lipstick core. In one embodiment of this invention, the coating of lip liner has a greater color value than the lipstick core. In another embodiment of this invention, the coating of lip liner has a lesser color value than the lipstick core.

Additional cosmetic materials may also be present in one or both the lipstick core and the lip liner. Suitable additional cosmetic materials include, for example, fragrances, flavorings, anti-chapping, shine enhancers, and/or dermatologic agents. The lip cosmetics may also contain a sunscreen to protect the skin (e.g., by blocking the skin from harmful ultraviolet light). Fragrances include, for example, the essential oils. Flavorings include, for example, methyl sucrose.

The lipstick core and the lip liner compositions of the present invention may include moisturizers. These moisturizers, which are hydrophilic, may be selected from the group consisting of polyhydric alcohols, ethoxylated and propoxylated polyols, and polysacharides. Preferred moisturizers include glycerine, panthenol, hexylene glycol, polyethylene glycol, polypropylene glycol, propylene glycol, sorbitol, L-pyroglutamic acid, sodium pyroglutamate, hydrolyzed animal collagen, hydrolyzed animal protein, and mixtures thereof. Most preferred is glycerine.

Shine enhancing agents include, for example, dimethicone-dimethicone copolyol, methyl silicone, and other dimethicones.

Dermatological agents that may also be present in one or both the lipstick core and the lip liner components of this invention include, for example, vitamins, antiinflammatory agents, hydroxy acids, and aloes. In a preferred embodiment, one or both of the lipstick core and lip liner components of the lip cosmetics of this invention comprise a dermatologic agent selected from the group consisting of vitamins, antiinflammatory agents, and hydroxy acids. Vitamins that may be used include, for example, Vitamin A, Vitamin D, and Vitamin E.

In another preferred embodiment, one or both of the lipstick core and lip liner components of the lip cosmetics of this invention include a sunscreen. Sunscreens that may be used include, for example, dioxybenzone, 2-ethylhexyl 2-cyano-3,3-diphenylacrylate, 2-ethylhexyl p-methoxycinnamate, 2-ethylhexyl salicylate, homosalate, menthyl anthranilate, oxybenzone, PABA and its derivatives, such as octyl dimethyl PABA, red petrolatum, titanium dioxide, and ferulic acid esters. Preferably, the sunscreen is selected from the group consisting of titanium dioxide, ferulic acid esters and preferably the sunscreen is titanium dioxide or ethyl ferulate or mixtures thereof.

The aesthetic and functional properties of both the lipstick core and the lip liner compositions may be controlled by a person skilled in the art by (a) varying the composition of the lipstick core and/or the composition of the outer layer of lip liner, and/or (b) varying the relative amounts of the lipstick core and lip liner compositions. The properties that may be controlled include, inter alia, the dryness, skin feel, color, and color value. In connection with varying the composition and relative amounts of the lipstick core and lip liner components, it should be kept in mind that in its intended use: (a) the lip cosmetic is applied to the outline of the lips in such a way that the outline of the lips is defined by the lip liner component of the lip cosmetic, and (b) the material that is applied to the body of the lips is, in effect, a mixture of the lipstick and lip liner components of the lip cosmetic.

The materials used in one or both the lipstick core and the lip liner compositions of this invention may also include other ingredients that are commonly employed by one of skill in the art in compositions for application to the skin (e.g., stabilizers, antimicrobial agents, antioxidants, preservatives, and the like). See, e.g., CFTA International Cosmetic Ingredient Dictionary, supra. Antioxidants include, for example, the propyl, octyl and dodecyl esters of gallic acid (e.g., propyl gallate), butylated hydroxyanisole, butylated hydroxytoluene, tocopherol (tocopheryl acetate), ascorbyl palmitate, and Vitamin E acetate; preservatives include, for example, butyl paraben, propyl paraben, ethyl paraben, methyl paraben, phenoxy ethanol, imidazolinyl urea, dimethyldimethoyl hydantoin, -(3-chloroallyl)hexaminium chloride, cetrimonium bromide, and trisodium ethylene diamine tetraacetic acid.

In one embodiment of this invention, the lipstick core composition comprises lanolin oil, castor oil, candelilla wax, lanolin, butyl stearate, oleyl alcohol, propyl gallate, propyl paraben, and tocopheryl acetate, and the lip liner composition comprises castor oil, lanolin oil, oleyl alcohol, ozokerite, candelilla wax, lanolin, carnauba, beeswax, mineral oil, propyl paraben, and propyl gallate. The amounts of the ingredients in this embodiment may be varied by a skilled practitioner to provide a lip cosmetic according to this invention.

Also provided herein are methods for preparing the lip cosmetic of the invention. In one embodiment, the method comprises:

providing a mold having an internal volume generally corresponding to the exterior shape of the lip cosmetic;

inserting into the mold a lipstick core mandrel having a shape generally corresponding to the shape of the lipstick core, leaving a lip liner volume;

introducing liquified lip liner composition into the lip liner volume and solidifying the lip liner composition to form what becomes the coating of lip liner;

removing the lipstick core mandrel from the mold leaving a lipstick core volume; and

introducing liquified lipstick composition into the lipstick core volume and solidifying the lipstick composition.

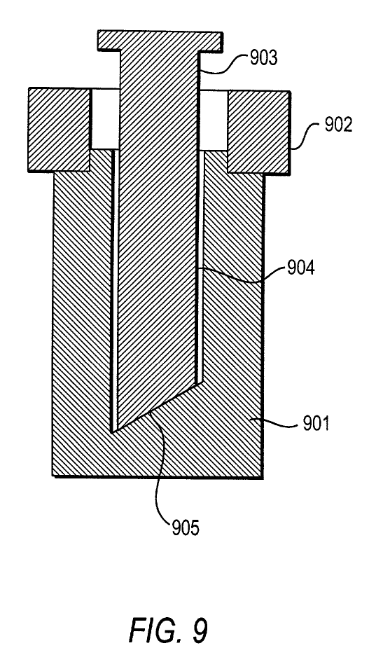

FIG. 9 shows, in cross section, a mold assembly that is illustrative of the type of mold assembly that may be used to practice the method described immediately above. In FIG. 9, the bottom portion of the mold assembly that defines the shape of the lip cosmetic is designated 901. The lipstick core mandrel that has a shape corresponding to the shape of the lipstick core is designated 903 and the upper portion of the mold assembly is designated 902. In use, the lipstick core mandrel 903 is inserted into the mold 901 as shown in FIG. 9, thereby defining lip liner volume 904. Liquified lip liner composition is then introduced into the lip liner volume and solidified to form what becomes the coating of lip liner.

The mold may then be opened and the lipstick core mandrel removed therefrom. When the mold is of the type that is opened and is closed again, there is left a lipstick core volume corresponding to the shape of the lipstick core mandrel. Liquified lipstick composition is introduced into the lipstick core volume and solidified to form the finished lip cosmetic. As can be seen in FIG. 9, the bottom 905 of the interior of the mold is shaped so that the applicator end of the lip cosmetic, as molded, is beveled.

Another embodiment of the method of the invention comprises:

providing a first mold having an internal volume generally corresponding to the lipstick core;

introducing liquified lipstick composition into the volume generally corresponding to the lipstick core and solidifying the lipstick composition to form the lipstick core;

providing a second mold having an internal volume generally corresponding to the lip cosmetic;

positioning the lipstick core into the internal volume generally corresponding to the lip cosmetic, leaving a lip liner volume; and

introducing liquified lip liner composition into the lip liner volume and solidifying the lip liner composition to form the coating of lip liner around the lipstick core.

A beveled end of suitable shape can be provided to the lip cosmetic by appropriately designing the mold(s) in the processes described above. Alternatively, the process may comprise a further step of beveling the applicator end of the lip cosmetic. Such a further step may be used when, for example, the lip cosmetic removed from the mold is bullet-shaped.

Alternatively, the invention provides a method for pouring a lip cosmetic directly into a casing. The casing serves as a mold having a volume generally corresponding to the volume of the desired lip cosmetic and may be used in place of corresponding molds in methods of the invention. This direct pour method does not require a separate step of mounting the lip cosmetic in a casing. In the direct pour method, either the lipstick core or the lip liner coating can be formed first. However, it is preferable to form the lipstick core first and then to form the lip liner coating around the core.

One embodiment of such a method according to the invention comprises:

providing a casing having an internal volume generally corresponding to the exterior shape of the lip cosmetic;

inserting into the casing a lip liner mandrel having a shape generally corresponding to the shape of the lip liner, leaving a lipstick core volume;

introducing liquified lipstick composition into the lipstick core volume and solidifying the lipstick composition;

removing the lip liner mandrel from the casing leaving a lip liner volume; and

introducing liquified lip liner composition into the lip liner volume and solidifying the lip liner composition to form the coating of lip liner around the lipstick core.

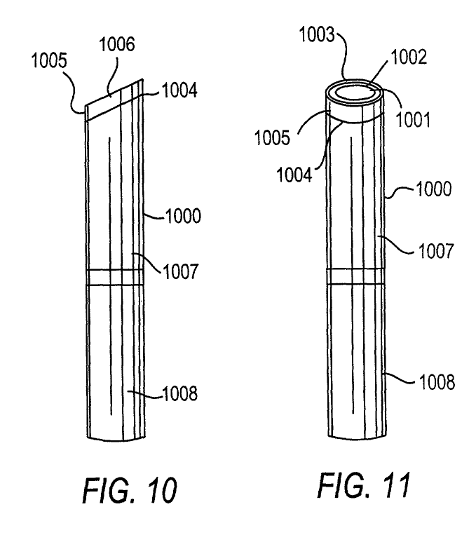

The direct pour method is particularly suited for preparing a lip cosmetic that is longer and slimmer than a conventional lip cosmetic. Casings for relatively long, slim lip cosmetics typically include a cup having a diameter of less than about 0.5 inches. A relatively long, slim lip cosmetic of the invention in a casing is depicted in FIGS. 10 and 11. Casing 1000 includes upper portion 1007 that holds the lip cosmetic. Lower portion 1008 holds a mechanism for moving the lip cosmetic out of the casing for application to the lips. Disposed within casing 1000 is the lip cosmetic with outer layer of lip liner 1002 and elongated lipstick core 1001.

As shown in FIG. 10, casing 1000 preferably has a sleeve 1005 that forms a volume 1006 that extends beyond top end 1004 of the casing (i.e., the end corresponding to the applicator end of the lip cosmetic). Sleeve 1005 aids in forming the lip cosmetic, and is conveniently removable after the lip cosmetic is formed. Preferably, the lip cosmetic initially extends beyond top end 1004, into volume 1006 defined by sleeve 1005.

As shown in FIG. 11, end 1003 of the lip cosmetic that is to be applied to the lips is beveled. Typically, sleeve 1005 is removed by any known method and any lip cosmetic extending into volume 1006 is beveled to conform to the shape of top end 1004, e.g., by clipping the applicator end of the lip cosmetic into the conforming shape. Sleeve 1005 may be removed and the lip cosmetic beveled, or the lip cosmetic may be beveled with sleeve 1005 in place, resulting in removal of sleeve 1005.

The lipstick and lip liner compositions used in the methods described above may be liquified by heating them to a temperature at which they are sufficiently flowable to be used in a molding process. Upon cooling in the mold, the compositions solidify to a state in which they may be removed from the mold.

The following non-limiting Examples 1 and 2 illustrate, respectively, a lip liner composition and a lipstick composition that may be used to make the lip cosmetic of the present invention.

### EXAMPLES

### Example 1

### Lip Liner Composition

LIP LINER COMPOSITION

| | | | |
|---|---|---|---|
| Product Name | INCI Name | % W/W | Supplier |

PHASE A

| | | | |
|---|---|---|---|
| Beeswax (White) | Beeswax | 5.00 | J.W. Hanson |
| | | | Co. |
| Candelilla Wax | Candelilla | 4.00 | J.W. Hanson |
| | Wax | | Co. |
| Carnauba Wax | Carnauba | 5.00 | J.W. Hanson |
| | | | Co. |
| Permethyl ® 102A | Isoeicosane | 5.00 | Presperse, |
| | | | Inc. |
| Permethyl ® 104A | Polyisobutene | 3.00 | Presperse, |
| | | | Inc. |
| Liponate IPP | Isopropyl | 8.00 | Lipo Chemicals |
| | Palmitate | | |
| Liponate TDTM | Tridecyl | 8.00 | Lipo Chemicals |
| | Trimellitate | | |
| Liponate 2-DH | PEG-4 | 4.00 | Lipo Chemicals |
| | Diheptanoate | | |
| Lipovol CO | Castor Oil | 32.85 | Lipo Chemicals |
| Oxynex L Liquid | Alcohol (and) | 0.05 | Rona/EM |
| | Tocopherol | | Industries |
| | (and) | | |
| | Vegetable Oil | | |
| | (and) | | |
| | Ascorbyl | | |
| | Palmitate | | |
| | (and) | | |
| | Ascorbic Acid | | |
| | (and) Citric | | |
| | Acid | | |
| Paraffin | Paraffin Wax | 4.00 | J.W. Hanson |
| | Fully Refined | | Co. |
| | 145 | | |

PHASE B

| | | | |
|---|---|---|---|
| SM-2000 | Mica (and) | 10.00 | Presperse, |
| | Silica | | Inc. |
| SP-29 UVS | Bismuth | 0.50 | Presperse, |
| | Oxychloride | | Inc. |
| | (and) Silica | | |
| | (and) Mica | | |
| Propylparaben, | Propylparaben | 0.10 | ISP |
| USP | | | |

PHASE C

| | | | |
|---|---|---|---|
| Titanium | Titanium | 3.00 | N/A |
| Dioxide (50% in | Dioxide | | |
| CO) | | | |
| Brown Iron | Iron Oxide | 7.50 | N/A |
| Oxide (50% in | | | |
| CO) | | | |

The above composition, when formulated by the procedure described below, would provide a firm formula suitable for use as the lip liner component in a lip cosmetic of this invention. The beeswax, candelilla wax, carnauba wax, and paraffin are used to provide structure to the composition. The paraffin is used to lend a firm structure to the composition. The Permethyl® 102A, Permethyl® 104A, Liponate TDTM, Liponate 2-DH, and Lipovol CO are emollients. SM-2000 and SP-29 UVS are used as feel modifiers. Propylparaben is a preservative. Titanium dioxide and iron oxide are colorants. This composition may be prepared as follows:

Procedure

1. In a suitable kettle equipped with lightnin type agitation, combine the Phase A ingredients in order. Stir the mixture well after the addition of each ingredient.

2. Heat the mixture to 80-85° C. and mix well until all the waxes have melted.

3. Add the Phase B ingredients in sequence, maintaining good agitation and temperature.

4. Mix the above ingredients to uniformity, while maintaining the heat for 10 minutes.

5. Add the pre-roller-milled Phase C ingredients into the mixture formed in step 4, in order, until no discreet particles are observed using Heggeman gauge.

6. Pour the mixture formed in step 5 into molds at 70-72° C.

### Example 2

### Red Lipstick Core Composition

RED LIPSTICK CORE COMPOSITION

| | | | |
|---|---|---|---|
| Product Name | INCI Name | % W/W | Supplier |

PHASE A

| | | | |
|---|---|---|---|
| Beeswax | Beeswax | 4.00 | N/A |
| (White) | | | |
| Candelilla Wax | Candelilla Wax | 3.00 | N/A |
| Carnauba Wax | Carnauba | 6.50 | N/A |
| Permethyl ® | Isoeicosane | 6.00 | Presperse, Inc. |
| 102A | | | |
| Permethyl ® | Polyisobutene | 4.00 | Presperse, Inc. |
| 104A | | | |
| Liponate TDTM | Tridecyl | 14.00 | Lipo Chemicals |
| | Trimellitate | | |
| Crystal “O” | Castor | 22.35 | CasChem |
| Castor Oil | (Ricinus | | |
| | Communis) Oil | | |
| Ceraphyl 847 | Octyldodecyl | 15.00 | ISP |
| | Stearoyl | | |
| | Stearate | | |
| Propylparaben, | Propylparaben | 0.01 | ISP |
| USP | | | |
| Tenox BHA | BHA | 0.05 | Eastman |
| | | | Chemical |
| Cashmir K-11 | Mica and | 2.00 | Presperse, Inc. |
| | Silica | | |
| Fionac MS-30C | Mica, Titanium | 5.00 | Presperse, Inc. |
| | Dioxide, and | | |
| | Iron Oxide | | |

PHASE B

| | | | |
|---|---|---|---|
| Titanium | Titanium | 4.00 | N/A |
| Dioxide - 50% | Dioxide and | | |
| in Castor Oil | Castor Oil | | |
| D&C Red No. 6 | Castor | 8.90 | N/A |
| Ba Lake in | (Ricinus | | |
| C.O. (33%) | Communis) Oil | | |
| | (and) D&C Red | | |
| | No. 6 | | |
| Brown Iron | Iron Oxide | 2.00 | N/A |
| Oxide - 50% in | (and) Castor | | |
| Castor Oil | Oil | | |
| D&C Red No. 7 | Castor | 3.10 | N/A |
| Ca Lake in | (Ricinus | | |
| C.O. (33%) | Communis) Oil | | |
| | (and) D&C Red | | |
| | No. 7 | | |

The above components, when formulated by the procedure described below, would provide a composition having a formula suitable for use as the lipstick core component of this invention. The beeswax, candelilla wax, and carnauba wax are used to provide structure to the composition. The Permethyl® 102A, Permethyl® 104A, Liponate IPP, Liponate TDTM, castor oil, and Ceraphyl R47 are emollients. Propylparaben is a preservative. BHA is an antioxidant. Cashmir K-II is used as a feel modifier. Flonac MS-30C, titanium dioxide, D&C Red No. 6, iron oxide, D&C Red No. 7 are colorants. The composition may be prepared as follows:

Procedure

Combine the Phase A ingredients in order and stir well after each addition. Mix well until uniform. Add Phase B ingredients in order. Start heating to 80-84° C. Mix well until uniform. Pour into molds at 80° C. +/−2° C.

While the present invention has been set forth in terms of specific embodiments thereof, it will be understood that the invention is defined by the appended claims.

I claim:

1. A lip cosmetic comprising:
(1) an elongated lipstick core; and
(2) an outer layer of lip liner around the periphery of the lipstick core, wherein the lipstick core comprises at least about 50% by weight of the lip cosmetic.

2. The lip cosmetic of claim 1 wherein the lip cosmetic and the lipstick core are cylindrical throughout at least the major portion of their length.

3. The lip cosmetic of claim 1 wherein the lip liner and lipstick are formulated so that in use the lip liner prevents or minimizes the tendency of the lipstick in the lip cosmetic to feather outside the border of the lips outlined by the lip liner.

4. The lip cosmetic of claims 1, 2 or 3 wherein the lipstick core and lip liner are present in amounts such that the lip cosmetic contains from about 50% to about 95% by weight of the lipstick core and from about 5% to about 50% by weight of the lip liner.

5. The lip cosmetic of claim 4 wherein the lipstick core and lip liner are present in amounts such that the lip cosmetic contains from about 85% to about 95% by weight of the lipstick core and from about 5% to about 15% by weight of the lip liner.

6. The lip cosmetic of claim 4 wherein the lipstick core and lip liner are present in amounts such that the lip cosmetic contains from about 50% to about 65% by weight of the lipstick core and from about 35% to about 50% by weight of the lip liner.

7. The lip cosmetic of claim 2 wherein the radius of the lip cosmetic is no more than about 1.4 times the radius of the lipstick core.

8. The lip cosmetic of claim 2 wherein the radius of the lip cosmetic is no more than about 1.2 times the radius of the lipstick core.

9. The lip cosmetic of claim 1 wherein the lipstick core has the same color as the outer layer of lip liner.

10. The lip cosmetic of claim 1 wherein the lipstick core has a different color than the outer layer of lip liner.

11. The lip cosmetic of claim 10 wherein the lip liner and lipstick are formulated so that in use the lip liner prevents or minimizes the tendency of the lipstick to feather outside the border of the lips outlined by the lip liner.

12. The lip cosmetic of claim 1 wherein the lipstick core has a lighter color than the outer layer of lip liner.

13. The lip cosmetic of claim 1 wherein the lipstick core has a darker color than the outer layer of lip liner.

14. The lip cosmetic of claim 1 wherein one or both of the lipstick core and lip liner includes one or more additional cosmetic materials, the additional cosmetic materials being selected from the group consisting of fragrances, sunscreens, dermatologic agents, and mixtures thereof.

15. A method for preparing a lip cosmetic comprising an elongated lipstick core and an outer layer of lip liner around the periphery of the elongated lipstick core comprising:
providing a mold having an internal volume generally corresponding to the exterior shape of the lip cosmetic;
inserting into the mold a lipstick core mandrel having a shape generally corresponding to the shape of the lipstick core, leaving a lip liner volume;
introducing liquified lip liner composition into the lip liner volume and solidifying the lip liner composition to form what becomes the coating of lip liner;
removing the lipstick core mandrel from the mold leaving a lipstick core volume; and
introducing liquified lipstick composition into the lipstick core volume and solidifying the lipstick composition.

16. The method according to claim 15 wherein the mold is designed to provide a beveled shape to the applicator end of the lip cosmetic.

17. A method for preparing a lip cosmetic comprising an elongated lipstick core and an outer layer of lip liner around the periphery of the lipstick core comprising:
providing a first mold having an internal volume generally corresponding to the lipstick core;
introducing liquified lipstick composition into the volume generally corresponding to the lipstick core and solidifying the lipstick composition to form the lipstick core;
providing a second mold having an internal volume generally corresponding to the lip cosmetic;
positioning the lipstick core into the internal volume generally corresponding to the lip cosmetic, leaving a lip liner volume; and
introducing liquified lip liner composition into the lip liner volume and solidifying the lip liner composition to form the coating of lip liner around the lipstick core.

18. The method according to claim 15 or claim 17 wherein the lipstick core and lip liner are present in amounts such that the lip cosmetic contains from about 50% to about 95% by weight of the lipstick core and from about 5% to about 50% by weight of the lip liner.

19. The method according to claim 15 or claim 17 wherein the lipstick core and lip liner are present in amounts such that the lip cosmetic contains from about 85% to about 95% by weight of the lipstick core and from about 5% to about 15% by weight of the lip liner.

20. The method according to claim 15 or claim 17 wherein the lipstick core and lip liner are present in amounts such that the lip cosmetic contains from about 50% to about 65% by weight of the lipstick core and from about 35% to about 50% by weight of the lip liner.

21. The method according to claim 17 wherein the first mold and the second mold are designed to provide a beveled shape to the applicator end of the lip cosmetic.

22. A method for applying a cosmetic to the lips comprising the step of applying to the lips the lip cosmetic of claim 1.

23. The method of claim 22 wherein the lip cosmetic is applied to the lips in such a way that the lip liner composition outlines the lips thereby preventing or minimizing the tendency of the lipstick composition that was applied to the lips to feather outside the boundaries of the lips.

24. The method of claim 22 wherein the lip cosmetic is applied to the lips in such a way that the lip liner composition highlights the outline of the lips.
